(19) United States
(12) Patent Application Publication
(10) Pub. No.: US 2021/0196179 A1
(43) Pub. Date: Jul. 1, 2021

(54) ELECTROCARDIOGRAPH LEAD WIRE

(71) Applicant: NINGBO XINWELL MEDICAL TECHNOLOGY CO., LTD, Ningbo, Zhejiang (CN)

(72) Inventors: Yubo ZHOU, Ningbo (CN); Jiewei YU, Ningbo (CN)

(73) Assignee: Ningbo Xinwell Medical Technology Co., Ltd, Ningbo, Zhejiang (CN)

(21) Appl. No.: 17/047,067

(22) PCT Filed: Sep. 12, 2018
(86) PCT No.: PCT/CN2018/105149

§ 371 Date: Dec. 23, 2020

(30) Foreign Application Priority Data
Apr. 13, 2018 (CN) 201810333665.2

(51) Int. Cl. A61B 5/282; A61B 5/30

(52) U.S. Cl. CPC A61B 5/282; A61B 2562/225; A61B 5/303

(57) ABSTRACT

Disclosed is an electrocardiograph (ECG) lead wire. The left electrode (120) and the right electrode (130) of the ECG lead wire are stacked and enclose an accommodation hole (101) for an electrode contact to pass through. At least one of the left electrode (120) and the right electrode (130) is made of an elastic material and/or is driven by an elastic member, so that the left electrode (120) and the right electrode (130) can move relative to each other under the action of an external force to widen the accommodation hole (101), and can move relative to each other under an elastic action force to narrow the accommodation hole (101). Since the size of the accommodation hole (101) can change, the electrode holder can be adapted to electrode contacts of various sizes on the market and makes same be firmly connected, thereby ensuring the stability and reliability of signal transmission.

### TECHNICAL FIELD

The present disclosure relates to electrocardiographic (ECG) devices, in particular to the structure of an ECG lead wire.

### BACKGROUND OF THE INVENTION

ECG monitors and ECG machines have become increasingly important medical monitoring and detecting equipment with the development of science and technology and the continuous improvement of medical standards. Generally speaking, host device, signal transmission features and signal acquisition features are the three important parts of ECG monitoring and detection. The signal acquisition features are usually connected by electrode holders with the signal transmission features. The electrode holders available on the market can be divided into several ways including clamp type, buckle type and banana-plug type. However, since the size of electrode contacts produced by different manufacturers is not uniform, it is easy to cause the problem of poor contact during an electrode contact produced by a certain manufacturer matching an electrode holder produced by other manufacturer. Consequently, it results in unstable or interrupted signal transmission, affecting the accuracy and stability of data of ECG monitoring and detection and also inconvenience to the operation of medical staff.

### SUMMARY OF THE INVENTION

The present disclosure provides a novel ECG lead wire.

An embodiment provided an ECG lead wire provided in an embodiment according to one aspect of the present disclosure may include:

an electrode holder which includes a connecting member, a left electrode and a right electrode, at least one of the left electrode and the right electrode being coupled to the connecting member, the left electrode and the right electrode being stacked and enclosed to form an accommodation hole for an electrode contact to pass through, at least one of the left electrode and the right electrode being made of an elastic material and/or driven by an elastic member, so that the left electrode and the right electrode can move relative to each other under an external force to widen the accommodation hole, and move relative to each other under an elastic force to narrow the accommodation hole, clamping the electrode contact;

a sleeve body which is wrapped outside the electrode holder, and at least a part of the left electrode and at least a part of the right electrode being exposed from the sleeve body; and

a connecting line which is communicated with the connecting member of the electrode holder for transmitting electrical signals.

As a further improvement of the ECG lead wire, the left electrode and the right electrode each are in a sheet structure, the left electrode has a first through hole, the right electrode has a second through hole, the first through hole and the second through hole are arrange in a stacked and staggered manner, so that the first through hole and the second through hole are partially overlapped, and the overlapped part forms the accommodation hole.

As a further improvement of the ECG lead wire, the left electrode and the right electrode each are in a linear structure.

As a further improvement of the ECG lead wire, a U-shaped body formed by the linear structure of the left electrode and a U-shaped body formed by the linear structure of the right electrode are arranged in a stagger manner, and the bottoms of the U-shaped bodies face inwards and are staggered to each other to enclose the accommodation hole.

As a further improvement of the ECG lead wire, the electrode holder has a first limiting structure which is configured to prevent the left electrode and the right electrode from being moved relative to each other in a direction that makes the accommodation hole smaller, thereby containing the minimum space of the accommodation hole.

As a further improvement of the ECG lead wire, the first limiting structure includes a first protrusion provided on the left electrode or the right electrode, and the first protrusion is correspondingly arranged on the moving path of the right electrode or the left electrode.

As a further improvement of the ECG lead wire, the electrode holder has a second limiting structure which is configured to prevent the left electrode and/or the right electrode from being moved in a direction that makes the accommodation hole wider, thereby containing the maximum space of the accommodation hole.

As a further improvement of the ECG lead wire, the second limiting structure includes a second protrusion provided on the left electrode or the right electrode, and the second protrusion is correspondingly arranged on the moving path of the right electrode or the left electrode.

As a further improvement of the ECG lead wire, the left electrode and the right electrode each are in a sheet structure, the left electrode has a first through hole and the right electrode has a second through hole, the edge of the second through hole protrudes toward the first through hole to form a limiting portion which is passed through the first through hole to confine the closest position where the left electrode and the right electrode approach each other and the farthest position where left electrode and the right electrode are away from each other.

As a further improvement of the ECG lead wire, the left electrode has a convex clamping portion which is passed through the limiting portion and extended into an area corresponding to the second through hole, and the clamping part and the wall of the second through hole are configured for clamping the electrode contact.

As a further improvement of the ECG lead wire, the connecting member includes a connecting body and two connecting legs coupled to the connecting body, and the left electrode and the right electrode each are connected to one connecting leg respectively.

As a further improvement of the ECG lead wire, the connecting member is in a substantially Y-shaped structure.

As a further improvement of the ECG lead wire, the connecting member includes a connecting body and a connecting leg coupled to the connecting body, the sleeve body forms two sleeve body legs arranged oppositely, one sleeve body leg is externally wrapped around the connecting leg, one of the left electrode and the right electrode is coupled to the connecting leg, and the other one is fixed on the sleeve body leg which does not wrap around the connecting leg.

As a further improvement of the ECG lead wire, the electrode holder has an anti-misplacing baffle which is extended to a gap outside the accommodation hole to avoid the wrong installation of the electrode contact.

As a further improvement of the ECG lead wire, the connecting member has a connecting groove in which the connecting wire is fixedly installed.

As a further improvement of the ECG lead wire, the accommodation hole enclosed by the left electrode and the right electrode is a closed hole structure.

As a further improvement of the ECG lead wire, the connecting member, the left electrode and the right electrode are made of conductive metal materials and formed integrally.

As a further improvement of the ECG lead wire, a label is provided on the sleeve body for guiding a medical staff to create the connection of the electrode contact according to the label.

According to the ECG lead wire of the aforesaid embodiments, the left electrode and the right electrode are arranged in a stacked manner to form an accommodation hole for the electrode contact to pass through, and at least one of the left electrode and the right electrode is made of an elastic material and/or driven by an elastic member, so that the left electrode and the right electrode can move relative to each other under the action of external force to widen the accommodation hole and can move relative to each other under an elastic force to narrow the accommodation hole. Since the size of the accommodation hole can be changed, the electrode holder can be adapted to electrode contacts of various sizes available on the market to make the electrode contacts be fixedly connected, thereby ensuring stable and reliable signal transmission.

### BRIEF DESCRIPTION OF THE DRAWINGS

FIGS. 1-2 are schematically structural diagrams of an ECG lead wire according to Embodiment 1 of the present disclosure;

FIG. 3 is a schematically structural diagram of the ECG lead wire and the connecting line of the embodiment shown in FIGS. 1-2;

FIG. 4 is a schematically structural diagram of an ECG lead wire according to Embodiment 2 of the present disclosure;

FIG. 5 is a schematically structural diagram of the ECG lead wire and the connecting line of the embodiment shown in FIG. 5;

FIGS. 6-7 are schematically structural diagrams of an ECG lead wire according to Embodiment 3 of the present disclosure;

FIG. 8 is a schematically structural diagram of the ECG lead wire and the connecting line of the embodiment shown in FIGS. 6-7;

FIG. 9 is a schematically structural diagram of an ECG lead wire according to Embodiment 4 of the present disclosure;

FIG. 10 is a schematically structural diagram of the ECG lead wire and the connecting line of the embodiment shown in FIG. 9;

FIG. 11 is a schematically structural diagram of an ECG lead wire according to Embodiment 5 of the present disclosure;

FIG. 12 is a schematically structural diagram of the ECG lead wire and the connecting line of the embodiment shown in FIG. 11;

FIG. 13 is a schematically structural diagram of an ECG lead wire according to Embodiment 6 of the present disclosure;

FIG. 14 is a schematically structural diagram of the ECG lead wire and the connecting line of the embodiment shown in FIG. 13;

### DETAILED DESCRIPTION

The present disclosure will be further described in detail below through specific embodiments with reference to the accompanying drawings. Common or similar elements are referenced with like or identical reference numerals in different embodiments. Many details described in the following embodiments are for the purpose of better understanding the present disclosure. However, those skilled in the art can realize with minimal effort that some of these features can be omitted in different cases or be replaced by other elements, materials and methods. For clarity some operations related to the present disclosure are not shown or illustrated herein so as to prevent the core from being overwhelmed by excessive descriptions. For those skilled in the art, such operations are not necessary to be explained in detail, and they can fully understand the related operations according to the description in the specification and the general technical knowledge in the field.

In addition, the features, operations or characteristics described in the specification may be combined in any suitable manner to form various embodiments. At the same time, the steps or actions in the described method can also be sequentially changed or adjusted in a manner that can be apparent to those skilled in the art. Therefore, the various sequences in the specification and the drawings are only for the purpose of describing a particular embodiment, and are not intended to be an order of necessity, unless otherwise stated one of the sequences must be followed.

The serial numbers of components herein, such as “first”, “second”, etc., are only used to distinguish the described objects and do not have any order or technical meaning. The terms “connected”, “coupled” and the like here include direct and indirect connections (coupling) unless otherwise specified.

### Embodiment 1

An ECG lead wire provided in this embodiment is used to connect a signal acquisition part and transmit electrical signals collected by the signal acquisition part to a host device.

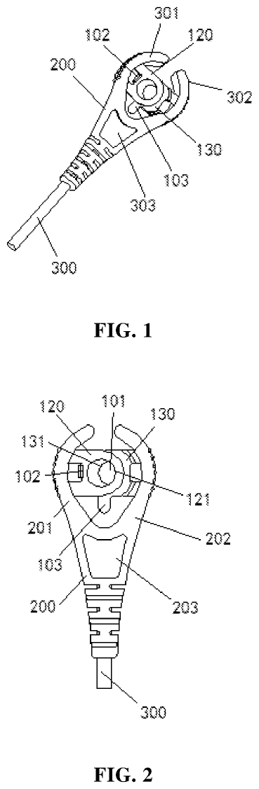

The ECG lead wire includes an electrode holder 100, a sleeve body 200 and a connecting line 300, please refer to FIGS. 1-3. The electrode holder 100 is used to directly contact an electrode contact and transfer the electrical signals collected by the electrode contact to the connecting line 300, and then to the host device for processing via the connecting line 300.

The electrode holder 100 includes a connecting member 110, a left electrode 120, and a right electrode 130. At least one of the left electrode 120 and the right electrode 130 is coupled to the connecting member 110 which can be used to connect with the connecting member 300 for transmission of electrical signals. The left electrode 120 and the right electrode 130 are stacked and enclose an accommodation hole 101 for the electrode contact to pass through. The wall of the accommodation hole 101 clamps the electrode contact by changing the diameter of the hole when fixing, so as to fix the electrode contact.

At least one of the left electrode 120 and the right electrode 130 is made of an elastic material and/or driven by an elastic member, so that the left electrode 120 and the right electrode 130 can move relative to each other by an external force to widen the accommodation hole 101 to accommodate the electrode contact. When the external force disappears, the left electrode 120 and the right electrode 130 can move relatively under an elastic force to reduce the accommodation hole 101 for clamping the electrode contact.

Since the size of the accommodation hole 101 can be changed, the electrode holder 100 can be adapted to electrode contacts of various sizes on the market. For example, the left electrode 120 and the right electrode 130 can be moved relatively by an external force to enlarge the size of the accommodation hole 101, so that electrode contacts with different sizes can be inserted into the accommodation hole 101. When the external force disappears, an elastic restoring force generated by the electrode holder 100 will reset the left electrode 120 and the right electrode 130 to narrow the accommodation hole 101 to clamp the electrode contact for firm connection, thereby ensuring stable and reliable signal transmission.

The sleeve body 200 is wrapped around the electrode holder 100 and the connecting line 300, wherein at least a part of the left electrode 120 and at least a part of the right electrode 130 are exposed from the sleeve body 200. The sleeve body 200 can wrap the electrode holder 100 and the connecting line 300 through injection molding, so that the electrode holder 100, the connecting line 300 and the sleeve body 200 become a whole, resulting in convenient manufacturing and processing and reliably firm connection between the electrode holder 100 and the connecting wire 300. The sleeve body 200 is usually made of an insulating material, which can not only ensure that the electrode holder 100 is disconnected from the outside, but also can make the connecting line 300 have a certain degree of toughness when swinging, avoiding damage to the wire sheath of the connecting line 300.

Please continue to refer to FIGS. 1-3. The accommodation hole 101 enclosed by the left electrode 120 and the right electrode 130 is a closed hole structure in an embodiment. The closed accommodation hole 101 is formed by the left electrode 120 and the right electrode 130 together. Compared with an open trough structure, this closed hole structure can ensure that the electrode contact is difficult to fall out of the accommodation hole 101 after being installed, which improves the convenience of an operator.

Further, please continue with FIGS. 1-3. In one embodiment, a sheet structure is adopted in the left electrode 120 and the right electrode 130. The left electrode 120 has a first through hole 121, and the right electrode 130 has a second through hole 131. The first through hole 121 and the second through hole 131 are arranged in a stacked and staggered manner so that the first through hole 121 and the second through hole 131 are overlapped at least partially to form the accommodation hole 101. When the left electrode 120 and the right electrode 130 are moved relatively, the overlapping portion of the first through hole 121 and the second through hole 131 will become larger or smaller, that is, the size of the accommodation hole 101 can be changed with the relative movement of the left electrode 120 and the right electrode 130 to adapt to electrode contacts having different sizes.

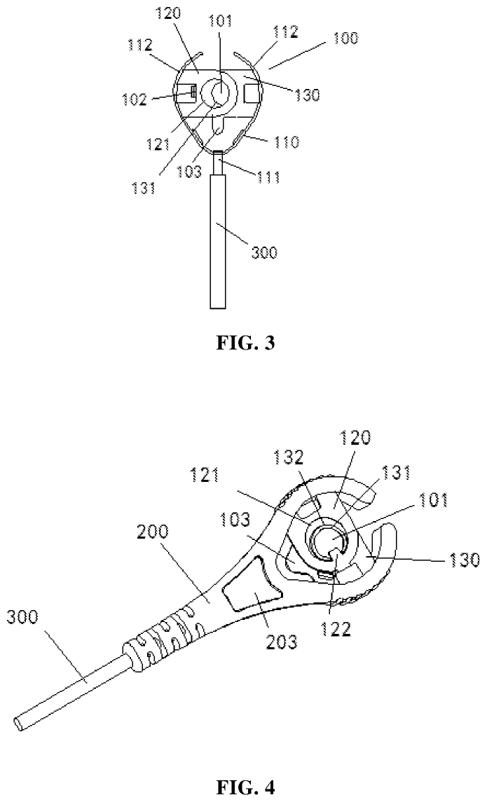

The connecting member 110, the left electrode 120 and the right electrode 130 are integrally formed of conductive metal materials in an embodiment, please refer to FIG. 3. In some other embodiments, the connecting member 110, the left electrode 120, and the right electrode 130, each being a separate structure, are finally connected as a whole by fixing.

Further, in some embodiments, the electrode holder 100 has a first limiting structure used to prevent the left electrode 120 and the right electrode 130 from moving relative to each other in a direction that makes the accommodation hole 101 smaller, which restrains the minimum space of the accommodation hole 101.

As shown in FIG. 3, the accommodation hole 101 will become narrower when the left electrode 120 and the right electrode 130 move to both sides, therefore the first limiting structure can be arranged at the direction in which at least one of the left electrode 120 and the right electrode 130 is moved to both sides; and when the left electrode 120 and the right electrode 130 is moved to the first limiting structure, it cannot continue to move, thereby limiting the accommodation hole 101 to no longer become narrower to avoid excessive deformation of the electrode holder 100, and also preventing the left electrode 120 and the right electrode 130 from being staggered too much to be unable to enclose the accommodation hole 101.

The first limiting structure includes a first protrusion arranged at the left electrode 120 or the right electrode 130 in some embodiments. The first protrusion is correspondingly arranged at the movement path of the right electrode 130 or the left electrode 120.

The first protrusion 102 is arranged on the right electrode 130, and is protruded toward the side at which the left electrode 120 locates and extended beyond the left electrode 120. The first protrusion 102 forms a limit on the left side of the left electrode 120. The left electrode 120 and the right electrode 130 will no longer move relative to each other when the left electrode 120 is moved to the left to a position at which the first protrusion 102 locates, and at this point, the accommodation hole 101 has the minimum size.

In addition, the electrode holder 100 has a second limiting structure used to prevent the left electrode 120 and/or the right electrode 130 from moving in a direction that makes the accommodation hole 101 larger in some embodiments, limiting the maximum space of the accommodation hole 101.

Similarly, the second limiting structure may include a second protrusion arranged on the left electrode 120 or the right electrode 130. The second protrusion is correspondingly disposed on the movement path of the right electrode 130 or the left electrode 120.

When the left electrode 120 and the right electrode 130 are moved to the middle, the accommodation hole 101 will become larger as shown in FIG. 3; therefore the second limiting structure can be arranged at the direction in which at least one of the left electrode 120 and the right electrode 130 is moved to the middle (although it is not shown in FIG. 3, it does not affect the understanding of those skilled in the art). When the left electrode 120 or the right electrode 130 is moved to the second limiting structure, it cannot continue to move, thereby limiting the enlargement of the accommodation hole 101 to avoid excessive deformation of the electrode holder 100, and also preventing the left electrode 120 and the right electrode 130 from being staggered too much to be unable to enclose the accommodation hole 101.

The first limiting structure and the second limiting structure may be arranged separately and independently, or may be connected as a whole (for example, forming a convex ring structure).

Further, the connecting member 110 includes a connecting body 111 and two connecting legs 112 coupled to the connecting body 111. The left electrode 120 and the right electrode 130 are connected to one connecting leg 112 respectively. The sleeve body 200 is sleeved on the connecting body 111 and the connecting legs 112, forming two sleeve body legs 201, 202.

The connecting member 110 is in a substantially Y-shaped structure. The connecting body 111 is a lower support part of the Y-shaped structure, and the two connecting legs 112 are respectively two branches of the Y-shaped structure. When the left electrode 120 and the right electrode 130 are moved relatively, the two connecting legs 112 are deformed accordingly. When the external force disappears, the connecting legs 112 provide elastic restoring force to help reset the left electrode 120 and the right electrode 130 (the left electrode 120 and the right electrode 130 per se may also have elastic restoring force).

The connecting member 110 has a connecting groove in which the connecting line 300 is fixedly installed. Specifically, the connecting groove may be arranged on the connecting body 111. The electrode holder 100 can be coupled to the connecting line 300 by riveting, improving the firmness and reliability of the connection.

On the other hand, the electrode holder 100 usually has many gaps to save cost and to adapt the shapes and movement of various components. It is easy for the operator to insert the electrode contacts into these gaps by mistake, resulting in wrong assembly of the electrode contacts. The electrode holder 100 has an anti-misplacing baffle 103 which is extended to the gap outside the accommodation hole 101 to prevent the electrode contact from being installed incorrectly in one embodiment, please refer to FIGS. 1-3.

The anti-misplacing baffle 103 and the right electrode 130 are integrally formed in FIGS. 1-3. In other embodiments, the anti-misplacing baffle 103 may also be arranged on other positions of the electrode holder 100, such as the left electrode 120, the connecting member 110. The anti-misplacing baffle 103 is arranged in a V-shaped gap of the Y-shaped connecting member 110 to block the V-shaped gap so that the electrode contact cannot be fitted into the gap in FIGS. 1-3.

Further, referring to FIGS. 1-2, a label 203 may be provided on the sleeve body 200 in one embodiment for guiding the medical staff to connect the electrode contact according to the label. The label 203 can be directly engraved on the mold by pasting or as an insert (displayed when the sleeve body 200 is injection molded) to guide the medical staff to connect a corresponding electrode contact according to the character of the label, improving the accuracy and effectiveness during operation.

### Embodiment 2

Provided in Embodiment 2 is another ECG lead wire.

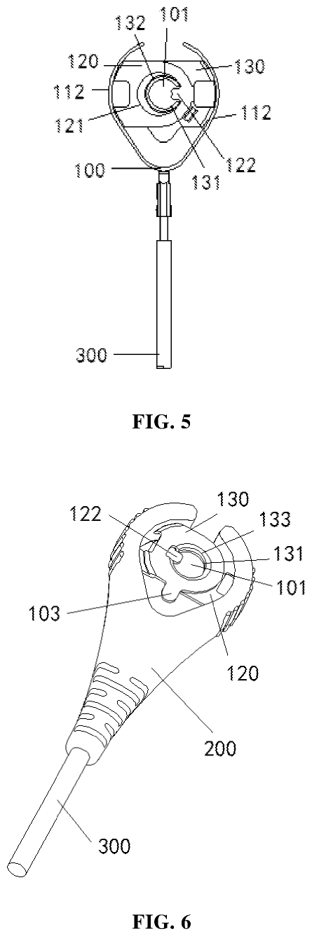

Please referring to FIGS. 4-5, the difference between the ECG lead wire shown in this embodiment and Embodiment 1 is that the left electrode 120 and the right electrode 130 each are in a sheet structure herein. The left electrode 120 has a first through hole 121, and the right electrode 130 has a second through hole 131. The second through hole 131 is arranged in the first through hole 121. The edge of the second through hole 131 is protruded toward the first through hole 121 to form a limiting portion 132. The limiting portion 132 is passed through the first through hole 121 to limit the closest position where the left electrode 120 and the right electrode 130 approach each other and the farthest position they are away from each other.

The limiting portion 132 may form a ring structure (closed or unclosed) and be arranged in the first through hole 121. Therefore, the limiting portion 132 has both the functions of the first limiting structure and the second limiting structure mentioned in the Embodiment 1, which not only simplifies the structure of the electrode holder 100, but also serves as a limiting function.

Further, referring to FIGS. 4-5, the left electrode 120 has a convex clamping portion 122 in an embodiment. The clamping portion 122 is passed through the limiting portion 132 and extended into an area corresponding to the second through hole 131. The clamping portion 122 and the wall of the second through hole 131 form the accommodation hole 101 for clamping the electrode contact. The clamping portion 122 is moved with the movement of the left electrode 120. When the clamping portion is moved, the length that it is extended into the second through hole 131 may also be changed accordingly, so the size of the accommodation hole 101 defined by the clamping portion and the second through hole 131 has also changed, which can be applied to electrode contacts of different sizes.

In addition, please refer to FIG. 4, the anti-misplacing baffle 103 may also be in a different shape from that in Embodiment 1.

### Embodiment 3

Provided in Embodiment 3 is still another ECG lead wire.

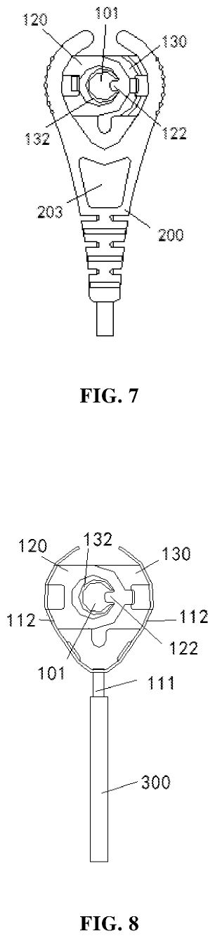

Please refer to FIGS. 6-8. The difference between the ECG lead wire shown in this embodiment and Embodiment 2 is that the wall of the second through hole 131 can be configured in an arc-shaped transition 133 having a large outer diameter and a small inner diameter. In this respect, the electrode contact is handily guided to be inserted into the second through hole 131, so that the connection therebetween can be realized by an operator with his/her hand without looking at them.

In addition, the anti-misplacing baffle 103 in the ECG lead wire provided in this embodiment is in a shape similar to that in Embodiment 1.

### Embodiment 4

Provided in Embodiment 4 is yet still another ECG lead wire.

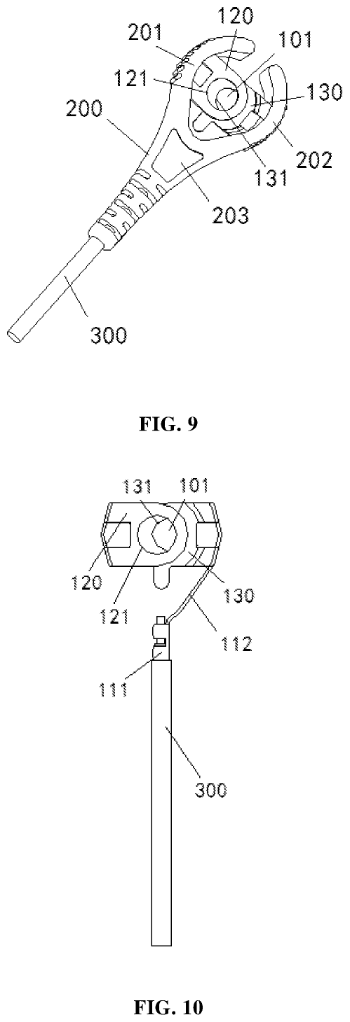

Please refer to FIGS. 9-10. The difference between the ECG lead wire shown in this embodiment and Embodiment 1 is that the connecting member 110 includes a connecting body 111 and one connecting leg 112 connected to the connecting body 111. The sleeve body 200 forms two sleeve body legs 201, 202 arranged oppositely. One sleeve body leg 202 is wrapped around the connecting leg 112; one of the left electrode 120 and the right electrode 130 is connected to the connecting leg 112, and the other one is fixed on the sleeve body leg 201 which does not wrap around the connecting leg 112.

Specifically, referring to FIG. 10, the right electrode 130 is fixed to the connecting leg 112 of the connecting member 110, and the left electrode 120 is fixedly connected to the sleeve body leg 201 of the sleeve 200. The sleeve body 200 per se has certain elasticity, so it can also provide elastic restoring force to the left electrode 120.

It is also naturally possible that the left electrode 120 is fixed to the connecting leg 112 of the connecting member 110, and the right electrode 130 is fixedly connected to the sleeve body leg 201 of the sleeve body 200.

### Embodiment 5

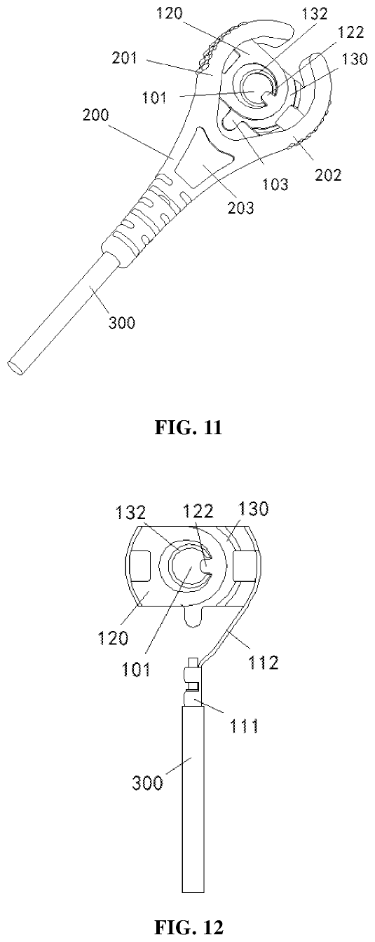

Please refer to FIGS. 11-12. The ECG lead wire shown in this embodiment is further improved on the basis of Embodiment 2. Specifically, the connecting member 110 includes a connecting member 111 and one connecting leg 112 coupled to the connecting member 111. The sleeve body 200 forms two sleeve body legs 201 and 202 arranged oppositely. A sleeve body leg 202 is wrapped around the connecting leg 112; one of the left electrode 120 and the right electrode 130 is connected to the connecting leg 112, and the other one is fixed on the sleeve body leg 201 which does not wrap around the connecting leg 112.

Please refer to FIG. 12. The right electrode 130 is fixed to the connecting leg 112 of the connecting member 110, and the left electrode 120 is fixedly connected to the sleeve body leg 201 of the sleeve body 200. The sleeve body 200 per se has certain elasticity, so it can also provide elastic restoring force to the left electrode 120.

It is also naturally possible that the left electrode 120 is fixed to the connecting leg 112 of the connecting member 110, and the right electrode 130 is fixedly connected to the sleeve body leg 201 of the sleeve body 200.

### Embodiment 6

Provided in Embodiment 6 is still yet another ECG lead wire.

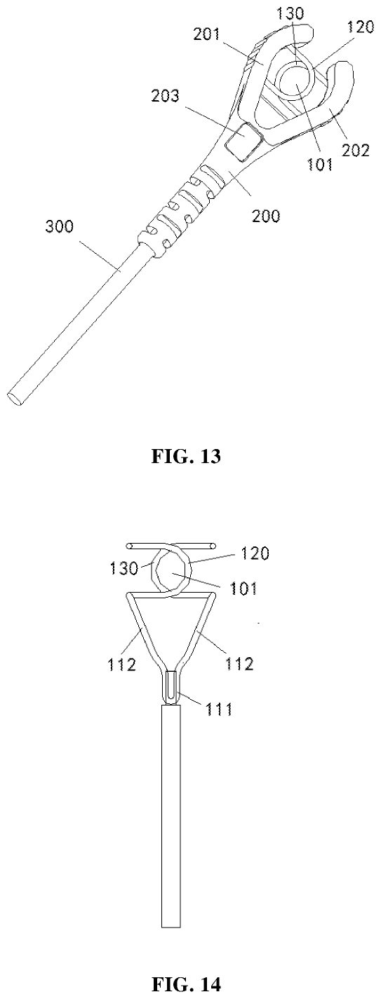

Please refer to FIGS. 13-14. The difference between the ECG lead wire shown in this embodiment and the aforesaid Embodiments 1 to 5 is that the left electrode 120 and the right electrode 130 of the electrode holder 100 each are in a linear structure. The left electrode 120 and the right electrode 130 of the linear structure are stacked and arranged together to form an accommodation hole 101 for the electrode contact to pass through. The wall of the accommodation hole 101 clamps the electrode contact by changing the diameter of the hole, so as to realize the fixing of the electrode contact.

Specifically, please refer to FIG. 14, in an embodiment, the linear structures of the left electrode 120 and the right electrode 130 respectively form a U-shaped body. The U-shaped body of the left electrode 120 and the U-shaped body of the right electrode 130 each are arranged in a layered manner in which their bottoms face inward and staggered to each other to enclose the accommodation hole 101.

The connecting member 110, the left electrode 120 and the right electrode 130 of the electrode holder 100 can be an integral whole made in a linear structure. The connecting member 110, the left electrode 120, and the right electrode 130 are made of conductive metal materials (such as a spring steel wire construction) which can not only transmit electrical signals, but also realize resetting by the elasticity of the steel wire.

The principle and implementation manners of the present disclosure has been described above with reference to specific embodiments, which are merely provided for the purpose of understanding the present disclosure and are not intended to limit the present disclosure. It will be possible for those skilled in the art to make variations based on the principle of the present disclosure.

1. An electrocardiographic (ECG) lead wire, comprising:
an electrode holder including a connecting member, a left electrode and a right electrode, at least one of the left electrode and the right electrode being coupled to the connecting member, the left electrode and the right electrode being arranged in a stacked manner and enclosed together to form an accommodation hole for an electrode contact to pass through, at least one of the left electrode and the right electrode being made of elastic material and/or driven by an elastic member, enabling the accommodation hole to be widen by the movement of the left electrode and the right electrode driven by an external force and to be narrowed by the movement of the left electrode and the right electrode driven by an elastic force, so as to fixedly clamp the electrode contact, wherein the electrode holder has a first limiting structure configured to prevent the left electrode and the right electrode from being moved relative to each other in a direction in which the accommodation hole becomes narrower, thereby limiting the minimum space of the accommodation hole;
a sleeve body externally wrapped the electrode holder, at least a part of the left electrode and at least a part of the right electrode being exposed from the sleeve body; and
a connecting line communicated with the connecting member of the electrode holder for transmitting electrical signals.

2. The ECG lead wire according to claim 1, wherein the left electrode and the right electrode each are in a sheet structure, the left electrode has a first through hole, and the right electrode has a second through hole, the first through hole and the second through hole are stacked and arranged in a staggered manner so as to partially overlap the first through hole and the second through hole, and the overlapped portion forms the accommodation hole.

3. The ECG lead wire according to claim 1, wherein the left electrode and the right electrode each are in a linear structure.

4. The ECG lead wire according to claim 3, wherein the linear structure of the left electrode and that of the right electrode form a U-shaped body respectively, and the U-shaped body of the left electrode and the U-shaped body of the right electrode are arranged in a layered manner in which their bottoms face inward and are staggered to each other to enclose the accommodation hole.

5. (canceled)

6. The ECG lead wire according to claim 1, wherein the first limiting structure comprises a first protrusion provided on the left electrode or the right electrode, and the first protrusion is correspondingly arranged on the moving path of the right electrode or the left electrode.

7. The ECG lead wire according to claim 1, wherein the electrode holder has a second limiting structure configured to prevent the left electrode and/or the right electrode from being moved in a direction in which the accommodation hole becomes wider, thereby limiting the maximum space of the accommodation hole.

8. The ECG lead wire according to claim 7, wherein the second limiting structure comprises a second protrusion provided on the left electrode or the right electrode, and the second protrusion is correspondingly arranged on the moving path of the right electrode or the left electrode.

9. The ECG lead wire according to claim 1, wherein the left electrode and the right electrode each are in a sheet structure, the left electrode has a first through hole and the right electrode has a second through hole, the edge of the second through hole protrudes towards the first through hole to form a limiting portion which is passed through the first through hole and configured to limit the closest position when the left electrode and the right electrode get close to each other and the farthest position when they are away from each other.

10. The ECG lead wire according to claim 9, wherein the left electrode has a protruding clamping portion which is passed through the limiting portion and inserted into an area corresponding to the second through hole, and the clamping portion and the wall of the second through hole are configured to clamp the electrode contact.

11. The ECG lead wire according to claim 1, wherein the connecting member comprises a connecting body and two connecting legs coupled to the connecting body, and the left electrode and the right electrode are coupled to one connecting leg respectively.

12. The ECG lead wire according to claim 11, wherein the connecting member is in a substantially Y-shaped structure.

13. The ECG lead wire according to claim 1, wherein the connecting member includes a connecting body and one connecting leg coupled to the connecting body, the sleeve body forms two sleeve body legs arranged oppositely, one sleeve body leg is externally wrapped around the connecting leg, one of the left electrode and the right electrode is coupled to the connecting leg, and the other one is fixed on the cover leg which is not wrapped around the connecting leg.

14. The ECG lead wire according to claim 1, wherein the electrode holder has an anti-misplacing baffle which is extended to a gap outside the accommodation hole to avoid wrong installation of the electrode contact.

15. The ECG lead wire according to claim 1, wherein the connecting member has a connecting groove in which the connecting line is fixedly installed.

16. The ECG lead wire according to claim 1, wherein the accommodation hole enclosed by the left electrode and the right electrode is a closed hole structure.

17. The ECG lead wire according to claim 1, wherein the connecting member, the left electrode and the right electrode are integrally formed of conductive metal materials.

18. The ECG lead wire according to claim 1, wherein a label is provided on the sleeve body to guide medical staff to create the connection of the electrode contact according to the label.
